(19) United States
(12) Patent Application Publication
(10) Pub. No.: US 2024/0398696 A1
(43) Pub. Date: Dec. 5, 2024

(54) DELIVERY OBJECTS SUITABLE FOR NEEDLE-FREE INJECTION EQUIPMENTS

(71) Applicants: PING-YEN CHUANG, HSINCHU CITY (TW); YUNG-LAN CHUANG, HSINCHU CITY (TW)

(72) Inventors: PING-YEN CHUANG, HSINCHU CITY (TW); YUNG-LAN CHUANG, HSINCHU CITY (TW)

(21) Appl. No.: 18/450,648

(22) Filed: Aug. 16, 2023

(30) Foreign Application Priority Data
May 30, 2023 (TW) 112120154

(51) Int. Cl. A61K 9/00; A61K 9/50

(52) U.S. Cl. CPC A61K 9/0021; A61K 9/5036; A61K 9/5052

(57) ABSTRACT

This invention provides a delivery object suitable for a needle-free injection equipment. A biodegradable material layer is used to encapsulate an active substance, forming an initial delivery object. Then, multiple initial delivery objects are placed within a carrier space formed by a delivery object carrier, creating a delivery object. The delivery object is placed within the needle-free injection equipment and accelerated by driving force provided by a power source, so that the delivery object has sufficient kinetic energy to be injected into the target object. The delivery object carrier and the biodegradable material layer will be consumed, decomposed, or metabolized within the target object, while releasing the active substance within the target object. Encapsulating the initial delivery object with the delivery object carrier will facilitate the acceleration of the delivery object so that the delivery object has sufficient kinetic energy to be injected into the target.

### CROSS-REFERENCE TO RELATED APPLICATIONS

This non-provisional application claims priority claim under 35 U.S.C. § 119 (a) on Taiwan Patent Application No. 112120154 filed May 30, 2023, the entire contents of which are incorporated herein by reference.

### TECHNICAL FIELD

This invention relates to a delivery object, particularly a delivery object suitable for a needle-free injection equipment, which can be used in gene guns, drug delivery systems, or biological ballistic delivery systems.

### BACKGROUND

Generally, medications, vaccines, or cosmetic materials are injected into a living organism by needle-based injection devices. During injection process, a needle of the needle-based injection devices will pierce the skin, and then delivers the liquid into the living organism. However, the needle-based injection devices have the following drawbacks: (1) Pain and discomfort associated with needle penetration through the skin; (2) Risk of infection, allergies, or redness at the injection site; (3) Potential for bleeding, stroke, or vision loss, especially when injecting in the triangular area of the face involving the eyes and nose; (4) Environmental pollution caused by the disposal of used needles; and (5) Risk of healthcare personnel contracting infections from used needles.

In order to improve the drawbacks of needle-based injection devices mentioned above, a needle-free injection equipment, such as gene guns, drug delivery systems, or biological ballistic delivery systems, has been developed. The needle-free injection equipment is used to inject a delivery object that comprises an active substance and metal particles into a target object, wherein the active substance is attached to the surface of the metal particles, such as tungsten particles, gold particles, or silver particles. The delivery object located in the needle-based injection device is accelerated by a power source, so that the delivery object has sufficient kinetic energy to penetrate cell membranes, cell walls, biological tissues (e.g., skin tissue), and/or organs (e.g., liver) and enter the target object. The active substance may be a medication, vaccine, biological substance, DNA, or RNA. Needle-free injection equipments have been widely used in genetic transformation of animals and plants, DNA vaccines, gene therapy, medical treatments, and medical aesthetics, among other fields.

As shown in FIG. 1, a conventional delivery object 10 suitable for the needle-free injection equipment includes an active substance 11 and metal particles 13. The metal particles 13 are carriers for the active substance 11, and the active substance 11 is attached to the outer surface of the metal particles 13. Due to the relatively large mass of the metal particles 13, they are advantageous for being accelerated by high-speed gas, such as nitrogen, allowing the metal particles to possess sufficient kinetic energy to penetrate biological tissues and enter the target object. However, during the process of penetrating biological tissues, the metal particles 13 may cause superficial damage to cells or biological tissues. Additionally, the metal particles cannot be degraded or metabolized within the target object and accumulate in the target object, causing harm to the target object. Furthermore, delivery objects made of metal particles have difficulty forming a suspended state within the needle-free injection equipment, thus hindering their uniform distribution both within the needle-free injection equipment and within the target object, ultimately affecting the efficacy of the active substance within the target object.

### SUMMARY

The primary objective of this invention is to provide a delivery object suitable for a needle-free injection equipment. A power source of the needle-free injection equipment can be used to accelerate a delivery object carrier of the delivery object, allowing the delivery object to have sufficient kinetic energy to enter the target object. The delivery object carrier can be engulfed, degraded, or metabolized in the target object, releasing an active substance within the target object. Furthermore, the delivery object of the invention does not require the use of metal particles and is advantageous in reducing damage to the target object.

To achieve the object, this disclosure provides a delivery object for a needle-free injection equipment comprising: a plurality of initial delivery objects including an active substance, and a biodegradable material layer encapsulating the active substance; and a delivery object carrier comprising a plurality of main carrier chains and a plurality of cross-linking chains, wherein the cross-linking chains connect with two or more main carrier chains to forming at least one carrier space between the main carrier chains and the cross-linking chains, and the initial delivery object is placed within the carrier space.

Thus, the delivery objects of the invention provide the following advantages:

The delivery object carrier can be engulfed, degraded, or metabolized within the target object without residue or harm.

The configuration of the delivery object carrier is advantageous in allowing the delivery objects to acquire sufficient kinetic energy from the needle-free injection equipment, enabling both an initial delivery object and the delivery object carrier to enter the target object.

By selecting suitable materials for the delivery object carrier and/or the biodegradable material layer, the degraded delivery object carrier and/or biodegradable material layer can form derivatives within the target object that possess cosmetic or medical effects.

By selecting the thickness or material of the biodegradable material layer, it is possible to control the time it takes for the biodegradable material layer to completely degrade within the target object. This allows the active substance within the delivery object to be released at different time points within the target object.

### BRIEF DESCRIPTION OF THE DRAWINGS

This disclosure will become more fully understood from the detailed description given herein below for illustration only, and thus not limitative of this disclosure, wherein:

FIG. 1 is a schematic diagram of a conventional delivery object suitable for a needle-free injection equipment.

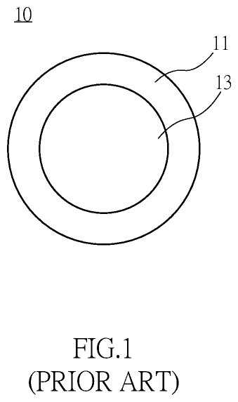

FIG. 2 is a schematic diagram of delivery objects suitable for a needle-free injection equipment according to an embodiment of the invention.

FIG. 3 is a schematic diagram of the individual structure of the delivery object according to an embodiment of the invention.

FIG. 4 is a schematic diagram of a needle-free injection equipment and the delivery objects according to an embodiment of the invention.

FIG. 5 is a structural diagram of the delivery object suitable for the needle-free injection equipment according another embodiment of the invention.

FIG. 6 is a structural diagram of the delivery object suitable for the needle-free injection equipment according another embodiment of the invention.

### DETAILED DESCRIPTION

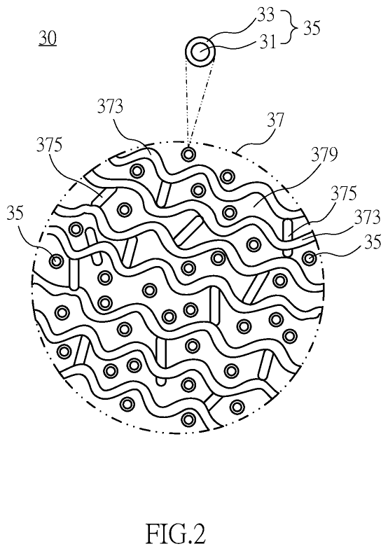

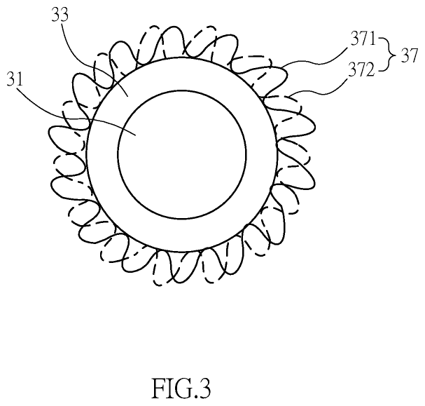

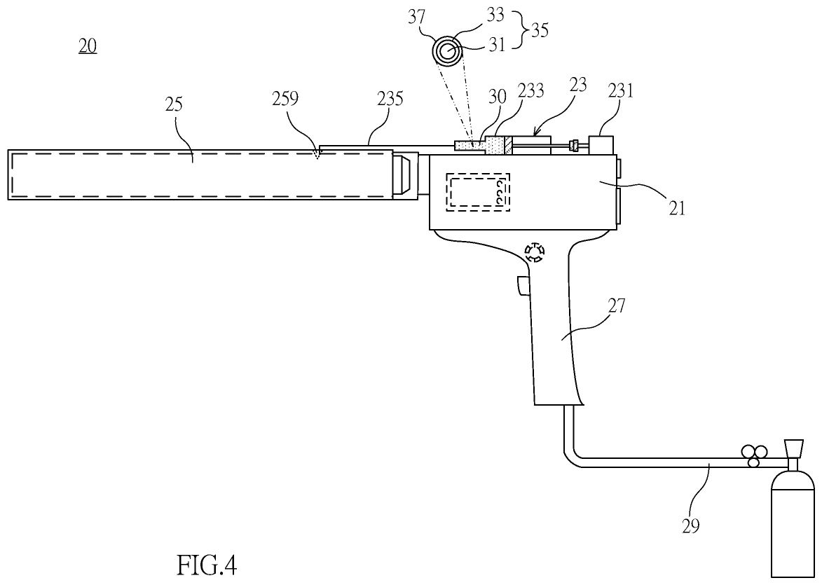

FIG. 2 is a schematic diagram of delivery objects suitable for a needle-free injection equipment according to an embodiment of the invention, FIG. 3 is a schematic diagram of the individual structure of the delivery object according to an embodiment of the invention, and FIG. 4 is a schematic diagram of a needle-free injection equipment and the delivery objects according to an embodiment of the invention. As shown in FIG. 4, the needle-free injection equipment 20 mainly comprises a pressure accumulator 21, a feeding device 23, an output tube 25, a grip 27, and a power source 29. The output tube 25 is connected to the front end of the pressure accumulator 21, while the grip 27 is connected to the bottom end of the pressure accumulator 21. The pressure accumulator 21 is connected to the power source 29. The power source 29 may be a gas supply device, an explosive device, or a vibration device, and is used to provide thrust into the output tube 25. For example, the power source 29 is a high-pressure gas cylinder. The feeding device 23 may be positioned at the top of the pressure accumulator 21 or as a separate standalone unit, detached from the pressure accumulator 21. The feeding device 23 may include a driver 231, a storage component 233, and a delivery tube 235. The storage component 233 may be a container, and is used to store delivery objects 30. The driver 231 may be a motor.

The output tube 25 is equipped with a feed inlet 259, which is connected to the delivery tube 235 of the feeding device 23, allowing the delivery objects 30 to be delivered through the delivery tube 235 and the feed inlet 259 into the output tube 25. The high-pressure gas provided by the power source 29 may be accelerated as it passes through the output tube 25, thereby driving the evenly distributed delivery objects 30 within the output tube 25. Thus, the high-speed delivery objects 30 is able to penetrate the surface layer, such as cellular bodies, tissues, or dermal layers, of the target object and enter the target object.

The delivery object 30 of this invention mainly consists of an active substance 31 coated with a biodegradable material, forming a biodegradable material layer 33 on the outer surface of the active substance 31 to create an initial delivery object 35. However, as the initial delivery object 35 is directly placed into the output tube 25 of the needle-free injection equipment 20 and propelled by the thrust generated by the power source 29. Due to the smaller mass of the initial delivery substance 35, it is unable to acquire sufficient kinetic energy to penetrate the surface of the target object and enter into it. Therefore, in this invention, multiple initial delivery objects 35 will be placed within a delivery object carrier 37.

The delivery object carrier 37 comprises multiple main carrier chains 373 (as shown by the curved lines in FIG. 2) and multiple cross-linking chains 375 (as shown by the cylindrical shapes in FIG. 2). The cross-linking chains 375 are used to connect two or more main carrier chains 373 to form carrier spaces 379 between multiple main carrier chains 373 and multiple cross-linking chains 375. The initial delivery object 35 can be placed within the carrier space 379. The combination of the initial delivery object 35 and the delivery object carrier 37 forms the delivery object 30 of the invention, which is suitable for use in the needle-free injection equipment 20.

In one embodiment of the invention, the delivery object carrier 37 may be hyaluronic acid (HA) or a glycuronic acid with a high molecular weight and/or long chain structure. The main carrier chains 373 may be hyaluronic acid chains, and the cross-linking agents 375 (also known as cross-linkers) may include, but are not limited to, diacids, polyols, divinylbenzene, diisocyanates, or methylenebisacrylamide (MBA). The hyaluronic acid chains 373 and the cross-linking agents 375 are able to form at least one three-dimensional carrier space 379, and at least one initial delivery object 35 can be placed within the carrier space 379.

Due to the compressible nature of delivery object carriers 37, such as hyaluronic acid, in an aqueous environment, the initial delivery material 35 can penetrate and adhere to the dissolved and expanded main carrier chains 373 of the delivery object carrier 37. When the delivery material 30 is subjected to high-speed gas impact provided by the power source 29, the delivery object carrier 37 and the initial delivery material 35 will be thoroughly atomized and accelerated. Together with supersonic gas, they will penetrate the surface layer of the target object and enter inside the target object.

The active substance 31 may include, but is not limited to, medications, aesthetic materials, vaccines, biological substances, genetic materials, or proliferants. For examples, the active substance 31 may includes dermal fillers, tissue growth promoters, botulinum toxin, skincare products, polymer particles, Vycross, DNA, RNA, proteins, cosmetic compositions, minerals, viral particles, botulinum toxin, hyaluronic acid, activating gels, collagen proteins, vitamins, cellulose, fruit acids, genetically modified organisms, essences, pearls, precious metals, pain relief genes, reducing agents, cold medicines, epidemic drugs, or anticancer drugs, and so on.

In one embodiment of the invention, the active substance 31 may be a proliferant, such as a cell preparation, an osmotic shock agent, an irritant or a chemotactic agent, a high-concentration platelet-rich plasma (PRP), stem cells, glucose, glycerol, zinc sulfate, phenol, tannic acid, creosote, pumice, cod liver oil acid, extracts, ozone, and/or vitamins. The target may be tendons, ligaments, dermal layers, joint capsules, cell bodies, scalp tissues, skin, spinal joints, skeletal muscles, etc., wherein the active substance 31 has significant therapeutic effects on sports injuries, degenerative joint arthritis, shoulder and neck pain, rotator cuff injuries, tennis elbow, elbow pain, wrist pain, knee pain, ankle pain, spinal pain, lower back pain, plantar fasciitis, meniscus injuries, cruciate ligament injuries, rotator cuff tendonitis, tendon disorders, chronic pain, skin scars, firming and smoothing of the skin, scalp follicle proliferation, hair loss prevention, and so on.

The biodegradable material layer 33 in the invention may be polyhydroxyalkanoates (PHA), polycaprolactone (PCL), polyglycolic acid (PGA), polylactic acid (PLA), poly-L-lactic acid (PLLA), or other special polymer materials, which can degrade within the target object. These materials exhibit excellent biocompatibility and their degradation rate can be controlled within the target object, organism or biological system.

In this invention, the initial delivery material 35 is placed within the delivery object carrier 37 to form the delivery object 30 with a larger mass. Therefore, the needle-free injection equipment 20 can accelerate the delivery object 30 and provide sufficient kinetic energy for it to penetrate the surface of the target object and enter inside. By using the delivery material 30 described in this invention, it is able to effectively reduce the damage caused by the penetration of the target object's surface using conventional metal particles 13.

Furthermore, both the delivery object carrier 37 and the biodegradable material layer 33 can be engulfed, degraded, or metabolized by the cells or tissues within the target object after entering, without leaving any residue. This effectively addresses the drawbacks of conventional delivery objects 10, wherein the metal particles 13 of the conventional delivery object 10 cannot be decomposed or metabolized in the target object. In one embodiment of the invention, by selecting suitable materials for delivery object carriers 37 and biodegradable material layers 33, substances such as collagen can be generated, which are beneficial for medical or cosmetic purposes.

In one embodiment of the invention, the delivery object carrier 37 may comprise a first delivery object carrier 371 and a second delivery object carrier 372. The first delivery object carrier 371 and the second delivery object carrier 372 may be composed of the same substance, different substances, or the same substance with different molecular weights. For example, both the first delivery object carrier 371 and the second delivery object carrier 372 are hyaluronic acid. Alternatively, the first delivery object carrier 371 is hyaluronic acid, while the second delivery object carrier 372 is a protein-containing substance. Another possibility is that the first delivery object carrier 371 is a high molecular weight hyaluronic acid with a molecular weight greater than 10,000 units, while the second delivery object carrier 372 is a low molecular weight hyaluronic acid with a molecular weight not exceeding 10,000 units.

In the experimental model of the invention, if the entire delivery object carrier 37 is composed of high molecular weight hyaluronic acid, such as 1×10⁷ amu (atomic mass unit), it will result in a high viscosity of the delivery object 30, which is not conducive to its aerosolization and uniform distribution within the output tube 25. Alternatively, if the entire delivery object carrier 37 is composed of low molecular weight hyaluronic acid, for example, 1×10³ amu, the needle-free injection equipment 20 may not be able to effectively accelerate the delivery material 30, resulting in insufficient kinetic energy for the delivery material 30 to enter the target object. Therefore, the ratio of the first delivery object carrier (high molecular weight hyaluronic acid) 371 to the second delivery object carrier (low molecular weight hyaluronic acid) 372 can be adjusted to generate a delivery object 30 with an appropriate viscosity. This ensures that the delivery substance 30 can obtain good atomization and injection kinetic energy from the needle-free injection equipment 20. Similarly, by combining different materials for the first delivery substance carrier 371 and the second delivery substance carrier 372, a delivery substance 30 with suitable viscosity can also be produced.

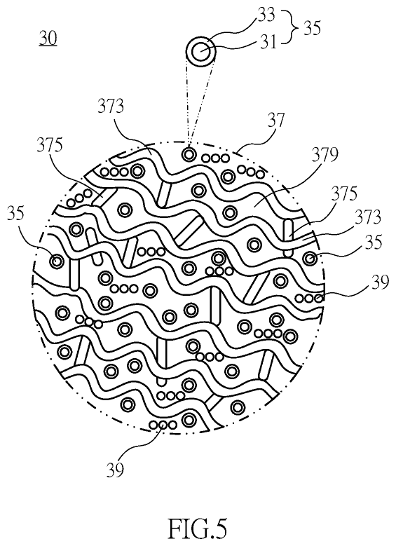

FIG. 5 is a structural diagram of the delivery object suitable for the needle-free injection equipment according an embodiment of the invention. The delivery object carrier 37 may be in the form of particle-type hyaluronic acid or gel-type hyaluronic acid. To facilitate injection into the output tube 25 and ensure uniform distribution, a buffer solution (Biological Buffers) 39 may be incorporated within the delivery object carrier 37. Examples of suitable buffer solutions include, but are not limited to, water, alcohols, or solutions containing active substances. More specifically, examples of buffer solutions include 1,3-Butylene Glycol, Tris(hydroxymethyl)aminomethane (TRIS), N-[Tris(hydroxymethyl)metyl]-3-aminopropanesulfonic acid (TAPS), or N-[Tris(hydroxymethyl)metyl]-2-aminopropanesulfonic acid (TES).

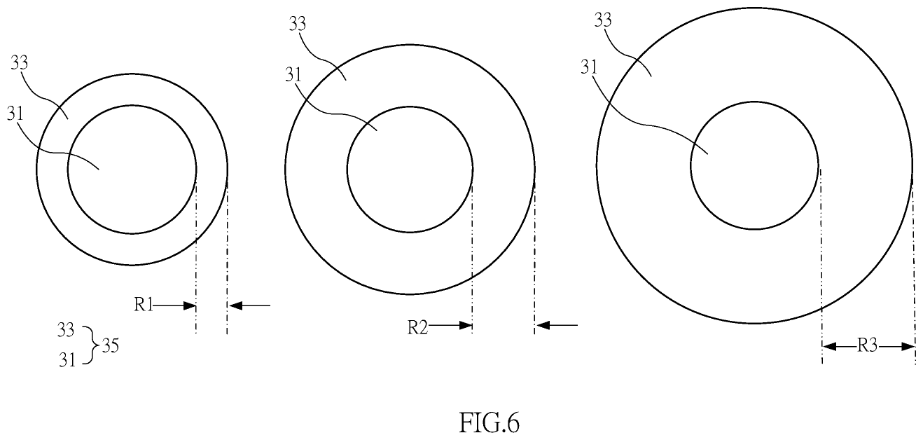

FIG. 6 is a schematic diagram of the initial delivery objects suitable for the needle-free injection equipment according to an embodiment of the invention. The biodegradable material layer 33 of the multiple initial delivery objects 35 may have different thicknesses, such as the first thickness R1, the second thickness R2, and the third thickness R3. The time required for the target object's cells or tissues to engulf, decompose, or metabolize the biodegradable material layer 33 of the initial delivery object 35 is related to, such as positive correlation, the thickness of the biodegradable material layer 33 and affects the timing of the release of the active substance 31 from the initial delivery object 35. In practical applications, the time required for complete decomposition of the biodegradable material layer 33 with different thicknesses can be used to calculate the time at which the active substance 31 in the target object takes effect, for example, 0.5 hours. In other words, the initial delivery object 35 in the invention can be used as a time-release medication.

In one embodiment of the invention, the delivery object 30 comprises multiple initial delivery objects 35, wherein the biodegradable material layer 33 of initial delivery objects 35 may have different thicknesses (R1, R2, R3), and they enter the target object together with the delivery object carrier 37. Due to the different thicknesses (R1, R2, R3) of the biodegradable material layer 33, the time required for the biodegradable material layer 33 of initial delivery objects 35 to be engulfed or decomposed may be different. As a result, the active substance 31 can be released at different time points within the target object.

For example, in the case of the initial delivery material 35 with the biodegradable material layer 33 of the first thickness R1, it may release the active substance 31 after one hour upon entering the target. In the case of the initial delivery object 35 with the biodegradable material layer 33 of the second thickness R2, it may release the active substance 31 one month after entering the target object. Similarly, in the case of the initial delivery object 35 with the biodegradable material layer 33 of the third thickness R3, it may release the active substance 31 two months after entering the target object. The initial delivery materials 35, which have three different thicknesses (R1, R2, R3), can be delivered into the target object by the needle-free injection equipment 20 in a single administration. Among them, the initial delivery material 35 with three different thicknesses (R1, R2, R3) will release the active substance 31 in the target object at different time points. This allows the initial delivery objects 35 with different thicknesses (R1, R2, R3) to release the active substance 31 at different time points within the target object. Through this embodiment of the invention, it is possible to extend or control the release time of the active substance 31 of the initial delivery object 35 within the target object. For example, a three-dose vaccine (active substance 31) can be simultaneously delivered into a user's body using a needle-free injection equipment 20, wherein each dose of the vaccine is released at different time points, reducing the frequency of vaccine administration and the time required for vaccination.

In one embodiment of the invention, the delivery object carrier 37 may be collagen, elastin, proteoglycans, or glycosaminoglycans (GAGs). The delivery object carrier 37 made from the those materials can also be used to carry the initial delivery object 35, and it facilitates the acquisition of sufficient kinetic energy for the initial delivery object 35 by the needle-free injection equipment 20.

In one embodiment of the invention, the active substance 31 may be a biodegradable material, and both the active substance 31 and the biodegradable material layer 33 can be either the same or different biodegradable materials. When the active substance 31 and the biodegradable material layer 33 enter the target object, they interact with macrophages and/or fibroblasts in the target object to generate collagen, which is beneficial for structural repair, wound healing, or tissue growth in the target object.

The above description is only a preferred embodiment of this disclosure, and is not intended to limit the scope of this disclosure. Modifications should be included within the scope of the patent application of this disclosure.

What is claimed is:

1. A delivery object for a needle-free injection equipment, comprising:
a plurality of initial delivery objects comprising an active substance, and a biodegradable material layer encapsulating the active substance to form; and
a delivery object carrier comprising a plurality of main carrier chains and a plurality of cross-linking chains, wherein the cross-linking chains connect with two or more main carrier chains to forming at least one carrier space between the main carrier chains and the cross-linking chains, and the plurality of initial delivery objects are located within the carrier space.

2. The delivery object according to claim 1, wherein the delivery object carrier is hyaluronic acid.

3. The delivery object according to claim 2, wherein the delivery object carrier is a gel-type hyaluronic acid or a granule-type hyaluronic acid.

4. The delivery object according to claim 1, wherein the active substance is a medication, a aesthetic material, a vaccine, a biological substance, a gene material, or a proliferant.

5. The delivery object according to claim 1, wherein the biodegradable material layer has a thickness, and the time for the biodegradable material layer to degrade within a target object is related to the thickness.

6. The delivery object according to claim 1, wherein the biodegradable material layer of the plurality of initial delivery objects within the carrier space has different thicknesses.

7. The delivery object according to claim 1, wherein the delivery object carrier is collagen, elastin, proteoglycan, or glycosaminoglycan.

8. The delivery object according to claim 1, further comprising a buffer solution located within the carrier space.

9. The delivery object according to claim 1, wherein the active substance is a biodegradable material.

10. The delivery object according to claim 9, wherein the active substance and the biodegradable material layer are made of the same material.

11. The delivery object according to claim 1, wherein the delivery object carrier comprises a first delivery object carrier and a second delivery object carrier, and the first delivery object carrier and the second delivery object carrier are made of different materials, or the same material with different molecular weights.

12. The delivery object according to claim 11, wherein the first delivery object carrier is hyaluronic acid, and the second delivery object carrier is a carrier containing protein substances.

13. The delivery object according to claim 11, wherein the first delivery object carrier is high molecular weight hyaluronic acid, and the second delivery object carrier is low molecular weight hyaluronic acid.

14. The delivery object according to claim 8, wherein the buffer solution is water, 1,3-Butylene Glycol, Tris(hydroxymethyl)aminomethane (TRIS), N-[Tris(hydroxymethyl)metyl]-3-aminopropanesulfonic acid (TAPS), or N-[Tris(hydroxymethyl)metyl]-2-aminopropanesulfonic acid (TES).
